(19) United States
(12) Patent Application Publication
(10) Pub. No.: US 2014/0364905 A1
(43) Pub. Date: Dec. 11, 2014

(54) ANCHOR SYSTEM AND DELIVERY DEVICE FOR USE THEREWITH

(71) Applicant: Richard Lunn, Kingston, MA (US)

(72) Inventors: Richard Lunn, Kingston, MA (US); David Paulk, Hopedale, MA (US)

(73) Assignee: Smith & Newphew ,Inc,, Memphis, TN (US)

(21) Appl. No.: 14/006,398

(22) PCT Filed: Mar. 22, 2012
(86) PCT No.: PCT/US2012/030095

§ 371 Date: Apr. 30, 2014

Related U.S. Application Data
(60) Provisional application No. 61/466,069, filed on Mar. 22, 2011.

(51) Int. Cl. A61B 17/04

(52) U.S. Cl. CPC A61B 17/0401; A61B 2017/0409; A61B 2017/044
USPC 606/232

(57) ABSTRACT

The present disclosure relates to an anchor system. The anchor system includes an anchor including a threaded outer portion and a non-threaded outer portion and a plug disposed within the anchor, the plug comprising a distal portion, a proximal portion, the proximal portion comprising a threaded portion and a coupling portion, and a through hole, wherein an inner surface of the threaded outer portion is shaped to interface with a portion of a delivery device and an inner surface of the non-threaded outer portion includes threads for engagement with threads on the threaded portion of the plug. A delivery device for use with the anchor system is also disclosed.

### CROSS-REFERENCE TO RELATED APPLICATIONS

This application is the U.S. National Stage of International Application No. PCT/US12/30095, filed on Mar. 22, 2012, which claims priority to and the full benefit of U.S. Provisional Application No. 61/466,069, filed on Mar. 22, 2011, the entire contents of which are incorporated herein by reference.

### BACKGROUND

1. Field of Technology

The present disclosure relates to devices and methods for repairing tissue.

2. Related Art

Arthroscopic procedures often require soft tissue to be reattached to bone. Originally, in order to achieve this, anchors were placed in the bone and sutures attached to the anchor were passed through the tissue to securely retain the tissue in place. Recently developed methods of reattaching tissue include attaching suture to tissue, placing the ends of the suture into a prepared bone hole, and inserting an anchor into the bone hole to provide fixation of the suture, and therefore the tissue, to the bone. Suture fixation via interference between the implant and the bone hole is dependent on patient bone quality. Therefore, if the patient bone is of poorer quality, there may be less interference between the anchor and the bone hole and, consequently less fixation of the suture and tissue to bone. Having an anchor and a method of fixating tissue to bone that reduces reliance on patient bone quality is needed.

### SUMMARY

In one aspect, the present disclosure relates to an anchor system. The anchor system includes an anchor including a threaded outer portion and a non-threaded outer portion and a plug disposed within the anchor, the plug comprising a distal portion, a proximal portion, the proximal portion comprising a threaded portion and a coupling portion, and a through hole, wherein an inner surface of the threaded outer portion is shaped to interface with a portion of a delivery device and an inner surface of the non-threaded outer portion includes threads for engagement with threads on the threaded portion of the plug.

In an embodiment, the threaded outer portion of the anchor includes longitudinal grooves. In another embodiment, the non-threaded outer portion is of a smaller diameter than the threaded outer portion. In yet another embodiment, the non-threaded outer portion is in engagement with the distal portion of the plug.

In another aspect, the present disclosure relates to a delivery device. The delivery device includes an outer shaft including a distal end, a proximal end, and a handle coupled to the proximal end, at least a portion of the distal end being of a smaller diameter than the proximal end; and an inner shaft disposed within the outer shaft, the inner shaft comprising a distal end, a proximal end, and a handle coupled to the proximal end, the distal end including prongs, an opening located between the prongs, and a marking In an embodiment, the delivery device further includes an anchor located on the distal end of the outer shaft and a plug located on the distal end of the inner shaft, the anchor including a threaded outer portion and a non-threaded outer portion, the plug including a distal portion, a proximal portion, the proximal portion including a threaded portion and a coupling portion, and a through hole, the coupling portion located within the opening of the inner shaft.

### BRIEF DESCRIPTION OF THE DRAWINGS

The accompanying drawings, which are incorporated in and form a part of the specification, illustrate the embodiments of the present disclosure and together with the written description serve to explain the principles, characteristics, and features of the disclosure. In the drawings:

FIG. 1 shows an isometric view of the anchor system and delivery device of the present disclosure.

FIG. 2 shows an isometric view of the anchor system and an end of the delivery device of FIG. 1.

FIG. 3 shows another isometric view of the anchor system and delivery device of FIG. 1.

FIG. 4 shows an isometric view of the anchor system and delivery device of FIG. 3.

FIGS. 5 and 6 show a method of tissue repair via use of the anchor system and delivery device of FIG. 1.

### DETAILED DESCRIPTION OF THE EMBODIMENTS

The following description of the preferred embodiment(s) is merely exemplary in nature and is in no way intended to limit the disclosure, its application, or uses.

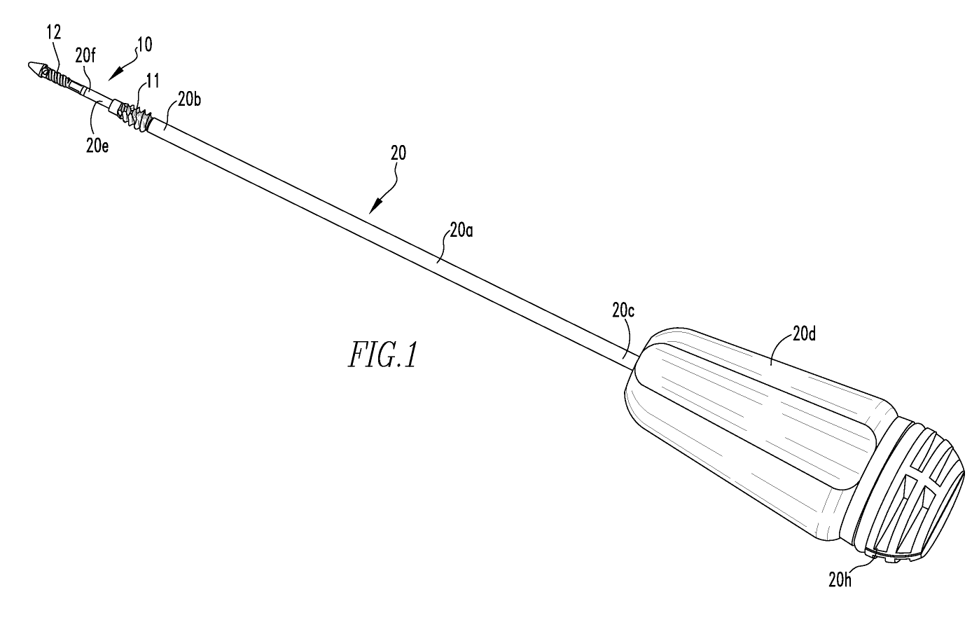

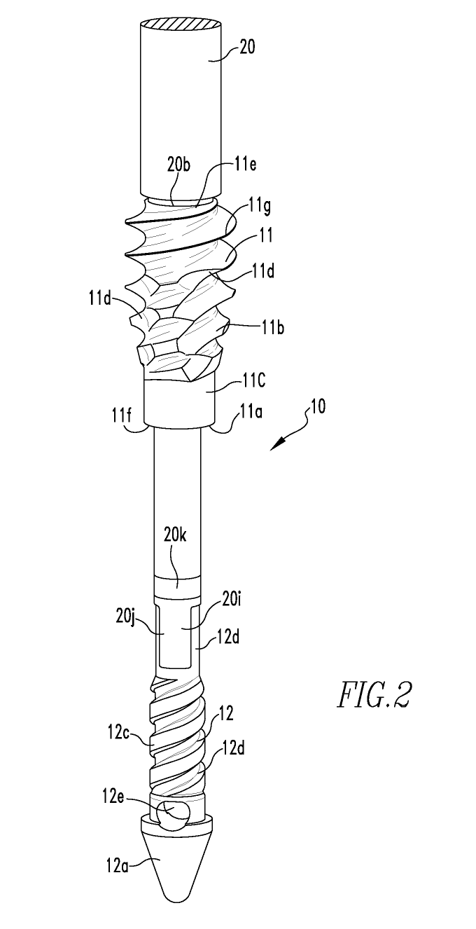

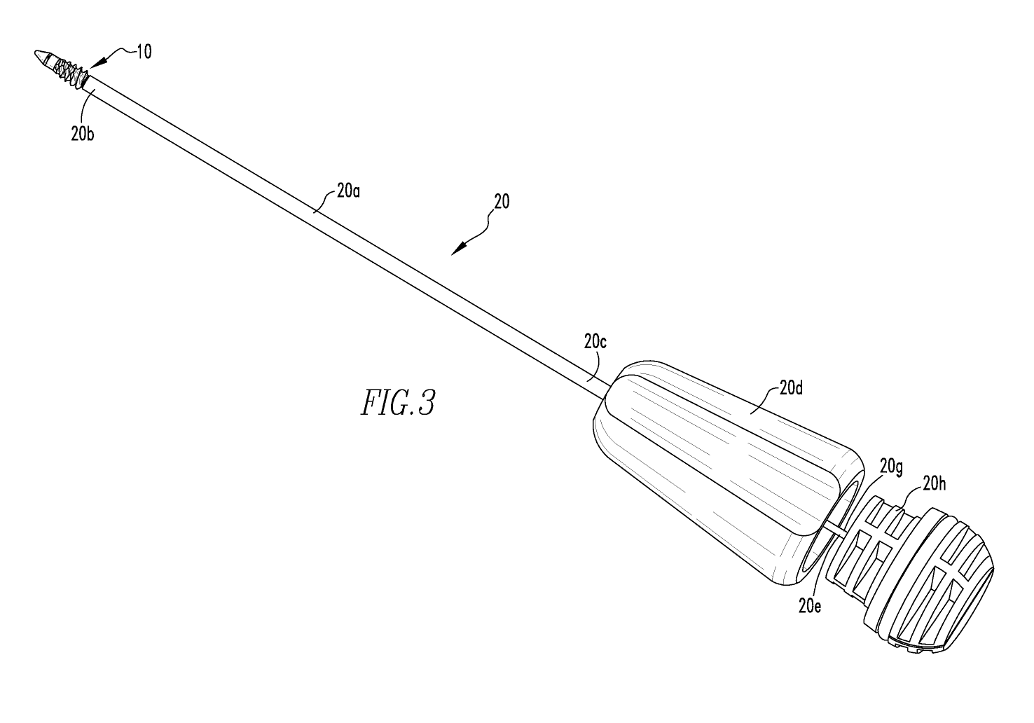

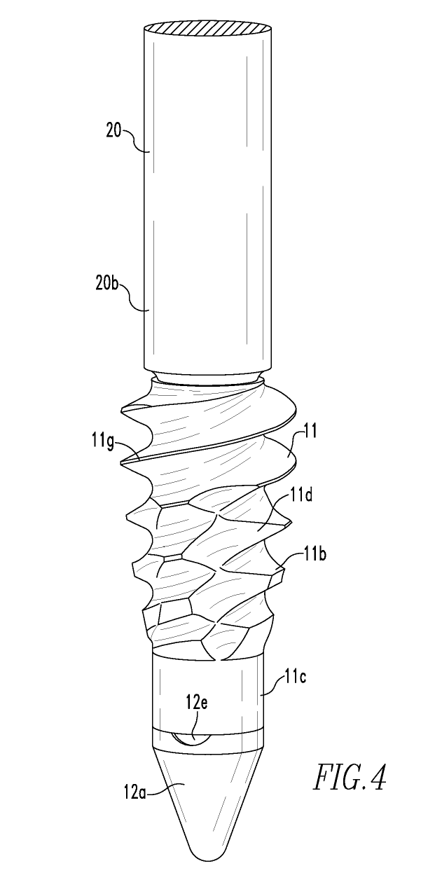

FIGS. 1-4 show the anchor system 10 and delivery device 20 of the present disclosure. The anchor system 10 includes an anchor 11 and a plug 12. The anchor 11 includes a cannulation 11a, a threaded outer portion 11b, and a non-threaded outer portion 11e. For the purposes of this disclosure, outer portion 11e is of a smaller diameter than threaded outer portion 11b. While threads 11g are located on the outer portion 11b, features other than threads may be used for allowing insertion of the anchor 11 into bone and fixation of the anchor 11 to the bone The threaded outer portion 11b includes longitudinal grooves 11d extending a partial length of the portion 11b. However, the grooves 11d may extend an entire length of the threaded outer portion 11b. The purpose of the grooves 11d is to allow the threads 11g to have more of a cutting action as the anchor 11 is inserted into bone. The inner surface 11e of outer portion 11b is shaped to mate with a portion of the delivery device 20, as will be explained later, and the inner surface 11f of outer portion 11c is shaped to mate with a portion of the plug 12, as will be further explained below. The plug 12 includes a distal portion 12a and a proximal portion 12b. The distal portion 12a is pointed, however, it is within the scope of this disclosure for the distal portion to be of a shape other than pointed. The proximal portion 12b includes a threaded portion 12c and a coupling portion 12d. It is within the scope of this disclosure for the proximal portion 12b to have features other than threads 12f. The plug 12 also includes a through hole 12e.

The delivery device 20 includes an outer shaft 20a having a distal end 20b and a proximal end 20c. A handle 20d is coupled to the proximal end 20c of the outer shaft 20a. The delivery device 20 also includes an inner shaft 20e disposed within the outer shaft 20a, the inner shaft 20e having a distal end 20f and a proximal end 20g. A handle 20h is coupled to the proximal end 20g of the inner shaft 20e. The distal end 20b of the outer shaft 20a is housed within the anchor 11 and is shaped to mate with the inner surface 11e. The distal end 20f of the inner shaft 20e includes prongs 20i and an opening 20j located between the prongs 20i. The coupling portion 12d is located within the opening 20j. Coupling means other than prongs 20i and coupling portion 12d may be used. The distal end 20f of the inner shaft 20e also includes a marking 20k, in the form a ring that encircles the end 20f, for purposes to be described later. Markings other than ring 20k may be used.

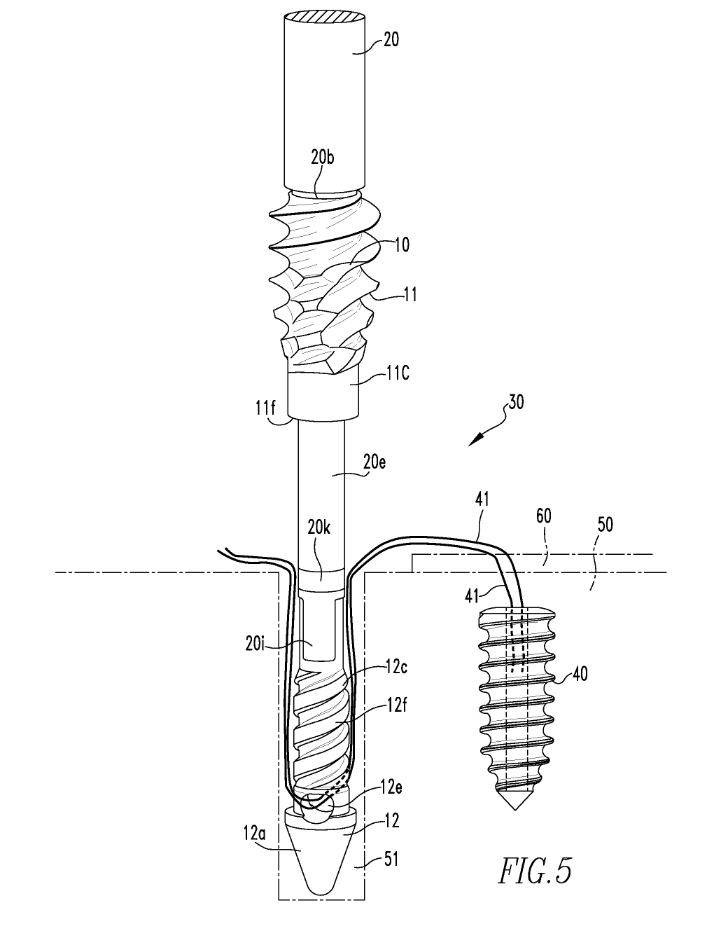

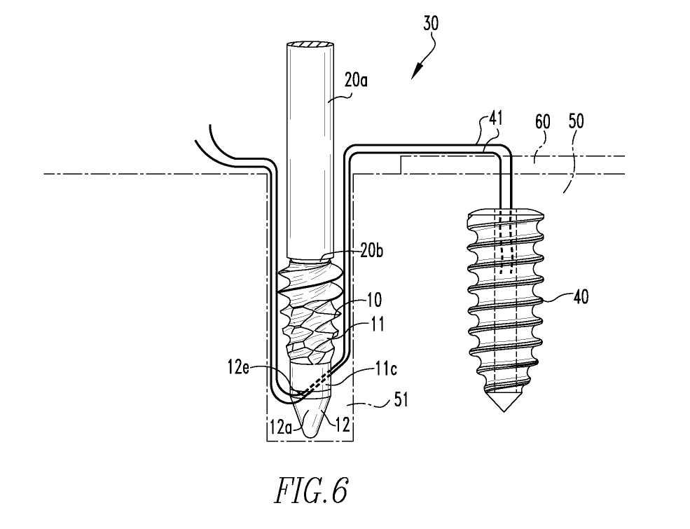

FIGS. 5 and 6 show a method 30 of tissue repair via use of the anchor system 10 and delivery device 20. A suture anchor 40 is inserted into bone 50, suture 41 is placed through tissue 60, and then the suture 41 is placed through the through hole 12e of the plug 12. The plug 12 is subsequently inserted into a previously created hole 51 in the bone 50 via use of the delivery device 20. Specifically, the user strikes handle 20h to axially drive the plug 12 into the hole 51. The plug 12 is driven into the hole 51 until the marking 20k is located within the bone 50, as shown in FIG. 5. The suture 41 may optionally be tensioned to pull the tissue 60 closer to the bone 50.

The outer shaft 20a is then moved axially via use of the handle 20d such that the outer portion 11c is brought into contact with the threaded portion 12c and threads (not shown) located on the inner surface 11f of the outer portion 11c are brought into engagement with the threads 12f of portion 12c. The inner surface 11f may include features other than threads to allow for coupling between the anchor 11 and the plug 12. The handle 20d of the outer shaft 20a may be rotated to rotate the outer shaft 20a and move the anchor 11 axially or the handle 20d may be used to move the outer shaft 20a and the anchor 11 axially without rotating. The handle 20d is then rotated to rotate the shaft 20a and the anchor 11. Rotating the anchor 11 causes the anchor 11 to move axially over the plug 12 until the outer portion 11c comes into contact with the distal portion 12a and the suture 41. The delivery device 20 is subsequently removed from the hole 12e. While portions of the suture 41 are still located between the anchor 11 and the bone hole 51, clamping of the suture 41 between the anchor 11 and the plug 12 provides the primary suture fixation force, which ends the sole dependence of suture fixation, and therefore tissue fixation, on patient bone quality.

For the purposes of this disclosure, the anchor 11 and the plug 12 both include a non-metal material, such as a polymer material. However, a metal material may be used. The polymer material is a non-absorbable polymer material, but may be an absorbable polymer material. Any absorbable polymer material may be used. An absorbable material that includes a filler, such as calcium sulfate, calcium carbonate, calcium phosphate, fibers, or other filler material that would provide for increased strength, osteoconductivity, decreased pH level of the polymer, or other benefits, may be used. The non-absorbable polymer material, may include, without limitation, polyetheretherketone (PEEK), or other non-absorbable polymer material. Likewise, the metal material may include stainless steel, titanium, or another metal material known to one of skill in the art.

The shafts 20a,20e of the delivery device 20 are of a biocompatible metal material, including, without limitation, stainless steel or titanium. However, other materials that would allow the delivery device 20 to withstand the forces placed on it during surgery may be used. The handles 20d,20h are of a non-metal material, including, without limitation, a polymer material. However, other materials may be used. The handles 20d,20h are coupled to the shafts 20a,20e via mechanical or non-mechanical methods. The marking 20k is placed on the shaft 20e via the use of a laser. However, other methods of placing the marking 20k on the shaft 20e may be used. The prongs 20i, as well as the decreased diameter section of shaft distal end 20b, may be made via the use of a process known to one of skill in the art.

As various modifications could be made to the exemplary embodiments, as described above with reference to the corresponding illustrations, without departing from the scope of the disclosure, it is intended that all matter contained in the foregoing description and shown in the accompanying drawings shall be interpreted as illustrative rather than limiting. Thus, the breadth and scope of the present disclosure should not be limited by any of the above-described exemplary embodiments, but should be defined only in accordance with the following claims appended hereto and their equivalents.

What is claimed is:

1. An anchor system comprising:
an anchor comprising a threaded outer portion and a non-threaded outer portion; and
a plug disposed within the anchor, the plug comprising a distal portion, a proximal portion, the proximal portion comprising a threaded portion and a coupling portion, and a through hole,
wherein an inner surface of the threaded outer portion is shaped to interface with a portion of a delivery device and an inner surface of the non-threaded outer portion includes threads for engagement with threads on the threaded portion of the plug.

2. The anchor system of claim 1 wherein the threaded outer portion of the anchor includes longitudinal grooves.

3. The anchor system of claim 1 wherein the non-threaded outer portion is of a smaller diameter than the threaded outer portion.

4. The anchor system of claim 1 wherein the non-threaded outer portion is in engagement with the distal portion of the plug.

5. A delivery device comprising:
an outer shaft comprising a distal end, a proximal end, and a handle coupled to the proximal end, at least a portion of the distal end being of a smaller diameter than the proximal end; and
an inner shaft disposed within the outer shaft, the inner shaft comprising a distal end, a proximal end, and a handle coupled to the proximal end, the distal end including prongs, an opening located between the prongs, and a marking.

6. The delivery device of claim 5 further comprising an anchor located on the distal end of the outer shaft and a plug located on the distal end of the inner shaft, the anchor comprising a threaded outer portion and a non-threaded outer portion, the plug comprising a distal portion, a proximal portion, the proximal portion comprising a threaded portion and a coupling portion, and a through hole, the coupling portion located within the opening of the inner shaft.
